(19) United States
(12) Patent Application Publication
(10) Pub. No.: US 2008/0063734 A1
(43) Pub. Date: Mar. 13, 2008

(54) Medical herb composition for the alleviation of cardiopulmonary ailments or the improvement of cardiopulmonary function in a mammal

(75) Inventors: Shuenn-Jyi Sheu, Taipei City (TW); Hsiao-Yin Yeh, Taipei City (TW); Wu-Che Wen, Danshuei Township (TW); Ching-Che Lin, Taipei City (TW); Chen-Ying Chiang, Taipei City (TW)

Correspondence Address: BACON & THOMAS, PLLC, 625 SLATERS LANE, FOURTH FLOOR, ALEXANDRIA, VA, 22314, US

(73) Assignee: Brion Research Institute of Taiwan, Taipei (TW)

(21) Appl. No.: 11/892,447

(22) Filed: Aug. 23, 2007

(30) Foreign Application Priority Data
Sep. 13, 2006 (TW) 095133815

(51) Int. Cl. A61K 36/258; A61P 11/00; A61P 9/00

(52) U.S. Cl. 424/728

(57) ABSTRACT

A medical herb composition for the alleviation of cardiopulmonary ailments or the improvement of cardiopulmonary function in a mammal is disclosed. The medical herb composition comprises: Schisandra extract; Ginseng extract; Radix ophiopogonis extract; and Saliva miltiorrhiza extract. The medical herb composition comprising the extracts above is proved to be efficient in enhancing cardiopulmonary function of a mammalian patient without heart disease, enforcing energy and raising strength. The medical herb composition is also capable of reducing the symptoms of breathing difficulties and stagnation of vital energy.

### BACKGROUND OF THE INVENTION

1. Field of the Invention

The present invention relates to a medical herb composition, more particularly, to a medical herb composition that can improve the cardiopulmonary function, strength, and the symptoms of breathing difficulties and stagnation of vital energy of a mammal.

2. Description of Related Art

Since many people seek health and nature, the Chinese herb-medicine industry has vigorously developed. Furthermore, owing to toxicity and side effects of “western” medicine, and drug resistance resulting from long-term administration, people have started to develop doubts about chemical drugs and thus constructively seek natural pharmaceuticals and therapy.

Chinese Herb-Medicine Information published by the Committee on Chinese Medicine and Pharmacy, Department of Health, Executive Yuan indicates: Schisandra is the dried ripe fruit of Schisandra chinensis (Turcz.) Baill. and its closely related plants, and Schisandra can function as kidney tonic and lung astringent, improve work performance, build strength, and help to reduce fatigue; Ginseng is the dried root of Panax ginseng C. A. Meyer, and the function of ginseng is to reinforce the vital energy, to remedy collapse and restore the normal pulse, to generate body fluids, to benefit the lungs, and to calm the nerves; Saliva miltiorrhiza is the dried root of Saliva miltiorrhiza BUNGE, and its function is to activate blood circulation and to transform stasis; and Radix ophiopogonis is the dried root of Ophiopogon japonicus KER-GAWLER and its closely related plants. The function of radix ophiopogonis is to clear away heart-fire, to moisten the lungs, to boost the stomach, and to generate body fluids.

Conventionally, the composition of Schisandra, Ginseng, and Radix ophiopogonis is used for the treatment of cardiovascular diseases. For example:

a. Shock: its clinical efficiency for acute obstructive suppurative cholangitis erupt acute DIC severe shock has been proved.

b. Acute myocardial infarction: it can enhance cardiac output, reduce peripheral vascular resistance, and improve the filling of important tissues and organs.

c. Heart failure: the composition with the effect similar to Cedi-lanid can improve the left heart function.

d. Irregular heart rhythms: it can improve sick sinus syndrome, premature beat, ventricular tachycardia, and so on.

e. Essential hypotension: according to the clinical research, essential hypotension can be improved efficiently by combined administration of Jin Kui Shen Qi Wan and the composition.

So far, the medical herb composition of Schisandra, Ginseng, and Radix ophiopogonis has often been used for treatment of the above conditions. However, no research indicates the composition can be used for daily heathcare. In addition, the known function of Saliva miltiorrhiza is to activate blood circulation and to transform stasis. However, less research has been performed on whether Saliva miltiorrhiza can raise the vitality of a mammal, such as a human.

### SUMMARY OF THE INVENTION

The present invention provides a medical herb composition of Schisandra, Ginseng, Radix ophiopogonis, and Saliva miltiorrhiza, and proves that the composition of the present invention exhibits the properties of improving the cardiopulmonary function of a mammalian patient without heart disease, raising the patient's strength and vitality. Also, the medical herb composition is capable of efficiently reducing the symptoms of breathing difficulties and stagnation of vital energy.

The medical herb composition of the present invention comprises Schisandra material, Ginseng material, Radix ophiopogonis material, and Saliva miltiorrhiza material.

The present invention further provides a medical herb composition, comprising Schisandra extract, Ginseng extract, Radix ophiopogonis extract, and Saliva miltiorrhiza extract.

The medical herb composition of the present invention can improve the cardiopulmonary function and vitality of a mammal, and reduce the symptoms of breathing difficulties and stagnation of vital energy.

The method for preparing the medical herb composition of the present invention can be any conventional preparation or extraction. Preferably, the method is concentration, water extraction, or alcohol extraction. More preferably, the method for preparing the medical herb composition of the present invention is a conventional method of preparation for Scientifically Concentrated Traditional Chinese Medicines.

The term “material” of the medical herb composition disclosed in the present invention refers to Chinese traditional herbs (including herbs of different plant basis but having the same efficacy, or common herbs used as substitutions), the extracts thereof, or the mixtures of the Chinese traditional herbs and the extracts thereof; the extracts can be water-based extracts or alcohol-based extracts.

Extracts in the medical herb composition of the present invention can be prepared by conventional methods of preparation for scientific herbs, preferably they are prepared by the method disclosed in the present invention. More preferably, the extracts are extracted by water, granulated by spray-drying, and then mixed proportionally with each other.

Preferably, the medical herb composition of the present invention further comprises a pharmaceutically acceptable carrier. For example, they can be used in orally administered medical herb compositions, the formulations including: capsules, tablets, emulsifiers, liquid suspensions, dispersants, and solvents. Taking tablets as an illustration, the commonly used carriers are galactose or cornstarch; lubricants and magnesium stearate are basic additives. When oral capsules are used, galactose and dried corn starch can act as effective diluting agents. Suitable sweetening agents, seasoning agents or pigments can be added if needed.

Preferably, the present invention can further comprise an excipient, which can be any kind known in the art, preferably corn starch.

### DETAILED DESCRIPTION OF THE PREFERRED EMBODIMENT

By conventional methods of preparation for Scientifically Concentrated Traditional Chinese Medicines including extraction, separation, concentration, and drying (as disclosed in Sun-Tien Medical Journal 4 and 5, 2005), extract powders of each plant were obtained.

The medical herb composition A used for the following embodiments comprises 6˜10 grams of Saliva miltiorrhiza extract, 1˜5 grams of Schisandra extract, 5˜9 grams of Ginseng extract, and 5˜9 grams of Radix ophiopogonis extract/per 12 gram of powders.

The amount of administration depends on the weight of the mammal to be treated. For a dog and a cat, the amount of daily administration is 1˜1.5 g/per 5 Kg. For a human, the amount of daily administration is 1.5˜2 g/per 10 Kg. Since the metabolisms of a dog and a cat are faster than that of a human, the amount of administration for a dog and a cat is about two-fold larger than that for a human.

The present invention provides the composition A for an older dog or a dog with slight heart disease, and observes the improvement of panting, coughing, and physical activity.

### Embodiment 1

The observed dog of the first embodiment was a male Shetland Sheepdog, 12 years old, intact, and of 8 Kg weight. Its clinical symptoms were poor heart function, slight panting, and heartbeat of 60 times per min.

After the administration of the composition A for 2 days, activity of the dog obviously improved. After 4 weeks of treatment, the symptom of panting disappeared, and the observed heartbeat rose to 72 times per min. In addition, its heart function of TR:98.6, AS:1.13 improved to TR:78.8, AS:0.88. According to the above results, the medical herb composition A of the present invention can efficiently improve the cardiopulmonary function and activity of an older dog.

### Embodiment 2

The observed dog of the second embodiment was a female Maltese, 12 years old and intact. Its clinical symptoms were poor heart function, panting, weariness, poor vitality and being unwilling to walk during the previous two years. After the administration of the composition A for 5 days, the dog's symptoms of panting and poor vitality improved, and it started to often walk back and forth at home.

According to the above results, the medical herb composition A of the present invention can improve the cardiopulmonary function an older dog.

### Embodiment 3

The observed dog of the third embodiment was a male Maltese, 12 years old and intact. Its clinical symptoms were poor heart function, panting, weariness, poor vitality and being unwilling to move (heart rate: 120-130 times per min). Ordinarily, the dog had digitalis, hypotensor drugs, and diuretic drugs continuously administered to efficiently control heart disease. However, its vitality and activity remained poor.

After the assistance of the composition A for two days (administration of digitalis, hypotensor drugs, and diuretic drugs once in the morning and the composition A once in the evening), the dog's symptoms of panting and poor vitality improved, and it started to often walk back and forth at home.

According to the above results, the medical herb composition A of the present invention can improve the cardiopulmonary function and raise the activity of an older dog.

### Embodiment 4

The observed dog of the fourth embodiment was a female part Dalmatian, 13 years old and intact. Its clinical symptoms were panting, coughing, a slight heart murmur, though not being infected with dirofilariaimmitis, and heart enlargement which was observed by X-ray. It also once fainted. Ordinarily, the dog had enapril, diuretic drugs, and heart assist drugs continuously administered to efficiently control heart disease. However, its vitality and activity remained poor.

After the assistance of the composition A for one week (administration of enapril, diuretic drugs, and heart assist drugs once in the morning and the composition A once in the evening), the dog's symptoms of panting and appetite improved.

According to the above results, the medical herb composition of the present invention can obviously improve the symptoms caused by heart disease of an older dog.

The results of the administration of the composition A for a human are described in the following. An older person or a person with slight heart disease usually exhibits the symptoms of poor strength, breathing difficulties, stagnation of vital energy, and poor vitality. After the administration of the composition A, those symptoms of the observed human were all found to have improved.

### Embodiment 5

The observed human of the fifth embodiment was a male of 170 cm height, 72 Kg weight, and without heart disease. His clinical symptoms were panting when stair climbing from the first to the third floor, breathing difficulties, and stagnation of vital energy.

After the administration of the composition A for 3 days, his cardiopulmonary function and the symptom of panting caused by stair climbing improved. During the continuous administration of the composition A, the symptoms of panting caused by stair climbing, breathing difficulties, and stagnation of vital energy all disappeared, and the weariness improved.

According to the above results, the medical herb composition A of the present invention can improve the cardiopulmonary function, strength, and the symptoms of breathing difficulties and stagnation of vital energy, and weariness.

### Embodiment 6

The observed human of the sixth embodiment was a female, 45 years old, 160 cm height, 50 Kg weight, and without heart disease. Her clinical symptoms were panting when stair climbing from the first to the fourth floor (she could not climb stairs to reach the fourth floor without having to rest many times during stair climbing), breathing difficulties and stagnation of vital energy.

After the administration of the composition A for 3 days, the symptom of panting caused by stair climbing several floors improved and disappeared. During that stair climbing, she no longer needed to take a rest, and her cardiopulmonary function improved. During the continuous administration of the composition A, the symptoms of panting caused by stair climbing, breathing difficulties, stagnation of vital energy, and the weariness all improved.

According to the above results, the medical herb composition of the present invention can improve the cardiopulmonary function, strength, and the symptoms of breathing difficulties, stagnation of vital energy, and wearness.

### Embodiment 7

The observed human of the present embodiment was a male, 53 years old, 165 cm height, 70 Kg weight, and without heart disease. His clinical symptom was panting when stair climbing from the first to the third floor.

After the administration of the composition A for 5 days, the symptom of panting caused by stair climbing floors improved. During the continuous administration of the composition A, the symptoms of panting caused by stair climbing, breathing difficulties, and stagnation of vital energy all improved and the weariness significantly decreased.

According to the above results, the medical herb composition A of the present invention can improve the cardiopulmonary function, and strength, and reduce weariness.

### Embodiment 8

The observed human of the eighth embodiment was a female, 50 years old, 154 cm height, 47 Kg weight, and without heart disease. Her clinical symptoms were weariness, breathing difficulties, and stagnation of vital energy.

After the administration of the composition A for 5 days, her vitality improved. During the continuous administration of the composition A, the symptoms of breathing difficulties and stagnation of vital energy all disappeared, and the weariness improved.

According to the above results, the medical herb composition of the present invention can improve strength, reduce weariness, and eradicate the symptoms of breathing difficulties and stagnation of vital energy.

### Embodiment 9

The observed human of the ninth embodiment was a female, 63 years old, 160 cm height, 55 Kg weight, and with low blood pressure. Her clinical symptoms were breathing difficulties, stagnation of vital energy, poor strength, and not being the capable of stair climbing from the first to the third floor without having to rest.

After the administration of the composition A for one day, the symptoms of breathing difficulties and stagnation of vital energy improved. After the administration of the composition A for 3 days, the symptoms of breathing difficulties and stagnation of vital energy all disappeared. Her strength improved, and she became able to climb stairs from the first to the third floor without having to rest. The weariness clearly had reduced. During the administration of a medicine for clods, her heart became unwell. However, when composition A was administered it was found that, the medicine for colds no longer caused her heart to be unwell. Thus, it is inferred that the composition A may present its efficiency.

According to the above results, the medical herb composition A of the present invention can improve the cardiopulmonary function and strength, reduce weariness, and the symptoms of breathing difficulties and stagnation of vital energy were eradicated.

### Embodiment 10

The observed human of the tenth embodiment was a female, 88 years old, 161 cm height, 43 Kg weight, heart hypertrophy, and arrhythmia controlled by the administration of western medicine. Her clinical symptoms were poor strength, breathing difficulties, and stagnation of vital energy.

After the administration of the composition A for 7 days, the symptoms of breathing difficulties and stagnation of vital energy reduced, and her strength improved.

According to the above results, the medical herb composition A of the present invention can improve the cardiopulmonary function and strength, and the symptoms of breathing difficulties and stagnation of vital energy were eradicated.

### Comparative Example 1

After the administration of the composition A for 2 months, the observed human of the embodiment 5 no longer received the composition A, and her symptoms which were the same as those before the administration of the composition A appeared again.

Alternatively, the observed human of the embodiment 5 received a composition B as a control group, comprising 5 grams of Schisandra extract, 10 grams of Ginseng extract, and 10 grams of Radix ophiopogonis extract/per 12 gram powders. After the administration of the composition B for 10 days, the cardiopulmonary function of the tested human improved. The symptom of panting when stair climbing improved by the administration of the composition B, but the efficiency of the composition B was less obvious than that of the composition A.

According to the results, the composition B can improve the cardiopulmonary function and strength, but the efficiency of the composition B is less obvious than that of the composition A. In addition, the composition B cannot obviously improve the symptoms of breathing difficulties and stagnation of vital energy.

### Comparative Example 2

After the administration of the composition A for 2 months, the observed human of the embodiment 6 no longer received the composition A, and her symptoms which were the same as those before the administration of the composition A appeared again.

Similarly, the observed human of the embodiment 6 then received a composition B as a control group, comprising 5 grams of Schisandra extract, 10 grams of Ginseng extract, and 10 grams of Radix ophiopogonis extract/per 12 gram powders. After the administration of the composition B for 14 days, the cardiopulmonary function of the tested human improved. The symptom of panting when stair climbing improved by the administration of the composition B, but the efficiency of the composition B was less obvious than that of the composition A. In addition, the composition B couldn't obviously improve the symptoms of breathing difficulties and stagnation of vital energy.

According to the results of the embodiments and comparative examples, the medical herb composition A of the present invention can efficiently improve the cardiopulmonary function, strength, and vitality of a mammal that does not have heart disease.

The efficiency of the composition A comprising Saliva miltiorrhiza extract is better than that of the composition B non-comprising Saliva miltiorrhiza extract. The composition A can efficiently raise the cardiopulmonary function and strength of a person to improve the symptoms of breathing difficulties and stagnation of vital energy of a mammal.

Although the present invention has been explained in relation to its preferred embodiment, it is to be understood that many other possible modifications and variations can be made without departing from the spirit and scope of the invention as hereinafter claimed.

What is claimed is:

1. A medical herb composition for the alleviation of cardiopulmonary ailments or the improvement of cardiopulmonary function in a mammal, comprising:
Schisandra extract;
Ginseng extract;
Radix ophiopogonis extract; and
Saliva miltiorrhiza extract.

2. The medical herb composition as claimed in claim 1, wherein the medical herb composition is used for improving the cardiopulmonary function of a mammal.

3. The medical herb composition as claimed in claim 1, wherein the medical herb composition is used for improving the symptoms of breathing difficulties.

4. The medical herb composition as claimed in claim 1, wherein the medical herb composition is used for improving the symptom of stagnation of vital energy.

5. The medical herb composition as claimed in claim 1, wherein the medical herb composition is prepared by extraction and concentration.

6. The medical herb composition as claimed in claim 5, wherein the concentration is a method introduced by Scientifically Concentrated Traditional Chinese Medicine.

7. The medical herb composition as claimed in claim 1, wherein the medical herb composition is prepared by water extraction, alcohol extraction, or the combination thereof.

8. The medical herb composition as claimed in claim 1, further comprising a pharmaceutically acceptable carrier.
